(19) United States
(12) Patent Application Publication
(10) Pub. No.: US 2004/0157334 A1
(43) Pub. Date: Aug. 12, 2004

(54) Method for determination of ethanol concentration in an aqueous solution containing an alcoholic beverage

(76) Inventors: Nikolay N. Barashkov, Hercules, CA (US); Boris M. Bolotin, Moscow (RU); Ben Zhong Tang, Kowloon (CN); Han Peng, Kowloon (CN); Junwu Chen, Guangzhou (CN)

Correspondence Address: MICHAELSON AND WALLACE, PARKWAY 109 OFFICE CENTER, 328 NEWMAN SPRINGS RD, P O BOX 8489, RED BANK, NJ, 07701

(21) Appl. No.: 10/638,952

(22) Filed: Aug. 11, 2003

Related U.S. Application Data
(60) Provisional application No. 60/402,306, filed on Aug. 12, 2002.

(51) Int. Cl. G01N 33/14

(52) U.S. Cl. 436/24

(57) ABSTRACT

A method is described for determining from a measurement of fluorescence intensity the concentration of ethanol in water. This end is attained by obtaining data which establishes a relationship between an intensity of fluorescence in the spectral region 490-650 nm for a solution obtained by mixing a fluorescent agent in a solvent, and an intensity of fluorescence in the spectral region 490-650 nm measured for a calibration sample obtained by mixing the fluorescent agent's solution with a water ethanol solution having a known concentration of ethanol.

### CLAIM TO PRIORITY

This application claims the benefit of our co-pending U.S. provisional patent application entitled “Fluorescent Method of Determinating Ethanol Content in Waster Solution” filed Aug. 12, 2003 and assigned Ser. No. 60/402,306, which is incorporated by reference herein.

### FIELD OF THE INVENTION

This invention relates to a method for the determination of the concentration of ethanol in an aqueous solution containing an alcoholic beverage. More specifically, the present invention relates to a method for determining ethanol concentration in such aqueous solutions by fluorescent spectroscopy.

### BACKGROUND OF THE INVENTION

The science of spectroscopy or spectral analysis is a well known method employed in qualitative and quantitative techniques for determining components and their concentration in a given sample.

Heretofore, spectroscopy has been used to ascertain the ethanol content of aqueous test samples, as for example, as employed in the brewing industry (see Fellows, T., In line alcohol and OG measurement using either infrared technology, Brewers Guardian, August 1993). Suitable analyzers for this purpose are available commercially, such as, for example, Model KSB Alcohol Analyzer marketed by McNab, Inc., Mount Vernon, N.Y.

These prior art techniques typically involve determining ethanol concentration in aqueous solutions by scanning absorption values of the sample over a range of wavelengths in the infrared region and then determining the ethanol concentration by evaluation of the significant peaks of the specific absorption of ethanol. Unfortunately, this technique requires a complicated evaluation means from the standpoint of both apparatus and methodology.

U.S. Pat. No. 5,679,955 describes a less complicated technique for determining ethanol concentrations in aqueous solutions. The patentees' procedure is based upon the provision of calibrating data which establishes a relationship between (a) a plurality of transmission values of an electromagnetic radiation in the near infrared region, measured a unique wavelength at which water is relatively opaque to the radiation while ethanol is relatively transparent thereto, of a plurality of calibration samples of the beverage containing ethanol in varying known concentrations, and (b) the known concentrations of ethanol in the calibration sample. In this manner, at least one light transmission value of the test sample at the unique wavelength, at which the calibration data were established is measured. The measured transmission value of the test sample is then transformed, by means of the relation established by the calibration data, into an indication of the concentration of ethanol in the test sample. Unfortunately, this technique provides the most reliable results at ethanol concentrations below about 50%, by volume, and typically in the range of up to about 20%, by volume with an optimum found to be below 10% by volume, at which point the change of absorbency is essentially proportional to the change of the ethanol concentration.

U.S. Pat. No. 5,470,755 relates to a fluorescent method for determining the alcohol content of a biological sample. This technique is based upon the fluorescent determination of the concentration of hemiacetals which are formed reversibly from an alcohol and corresponding keto compound. This reaction may be effected in sensors which are used for optical determination of alcohols. Although this technique has been used, it is limited in that the procedure is relatively complicated and involves the use of polymer membranes containing a keto compound embedded in a polymeric material.

In light of the limitations of the known prior art techniques, workers in the art have continued their quest in search of new techniques which eliminate the prior art deficiencies.

### SUMMARY OF THE INVENTION

In accordance with the present invention, this end has been attained by the use of a novel method for determining the concentration of ethanol in an aqueous sample conducted in a broad range of ethanol concentration ranging from 0 to 80.0 volume %.

More specifically, the present invention is premised upon the concept that certain fluorescent compounds evidence a unique type of solvatochromism. For example, it has been found that the aggregated form of fluorescent agent, 1-methyl-1,2,3,4,5 pentaphenylsilole, (MPPS), exhibits a strong emission in solid state and a weak emission in certain solvents, such as ethanol wherein this compound is molecularly dissolved.

### BRIEF DESCRIPTION OF THE DRAWINGS

The invention will be more readily understood by reference to the following detailed description taken in conjunction with the accompanying drawing wherein:

FIG. 1 is a graphical representation on coordinates of content of ethanol in volume % against quantum yield showing the dependence of fluorescence quantum yield of compound III in an ethanol-water mixture on ethanol content;

FIG. 2 is a graphical representation on coordinates of content of ethanol in volume % against quantum fluorescent intensity in relative units showing the dependence of fluorescence intensity of compound I (R=H) at 530 nm. on ethanol content in a water-alcohol component (N-methylpyrrolidone-ethanol-water mixture);

FIG. 3 is a graphical representation on coordinates of content of ethanol in volume % against quantum fluorescent intensity in relative units showing the dependence of fluorescence intensity at 644 nm. of compound II (R═CH₃) on ethanol content in a water-alcohol component (N-methylpyrrolidone-ethanol-water mixture);

FIG. 4 is a graphical representation showing the fluorescence spectra in nanometers of a solution of Lumogen Yellow S 0790 (starting concentration in N-methylpyrrolidone (0.13%) after the addition of water alcohol mixtures with ethanol concentrations ranging from 0 top 30 volume % (ratio between N-methylpyrrolidone and ethanol-water component being 3.0:0.7 w/w);.

FIG. 5 is a graphical representation showing the fluorescence spectra in nanometers of a solution of Lumogen Yellow S 0790 (starting concentration in N-methylpyrrolidone (0.13%) after the addition of three test samples of alcoholic beverages (ratio between N-methylpyrrolidone and the test sample being 3.0:0.7 w/w);

FIG. 6 is a graphical representation showing the fluorescence spectra in nanometers of a solution of 5-(4-dimethylaminobenzylidene)-barbituric acid (starting concentration in N-methylpyrrolidone being 0.13%) after the addition of water alcohol mixtures having an ethanol concentration ranging from 40 to 80 volume % (the ratio between N-methylpyrrolidone and the ethanol water component being 3.1:1.0 w/w); and

FIG. 7 is a graphical representation showing the fluorescence spectra in nanometers of a solution of compound III (1-methyl-1,2,3,4,5-pentaphenylsilole) after the addition of water alcohol mixtures with ethanol concentrations ranging from 20% to 50%, the ratio between ethanol and ethanol water component being 1:9 v/v).

### DETAILED DESCRIPTION OF THE INVENTION

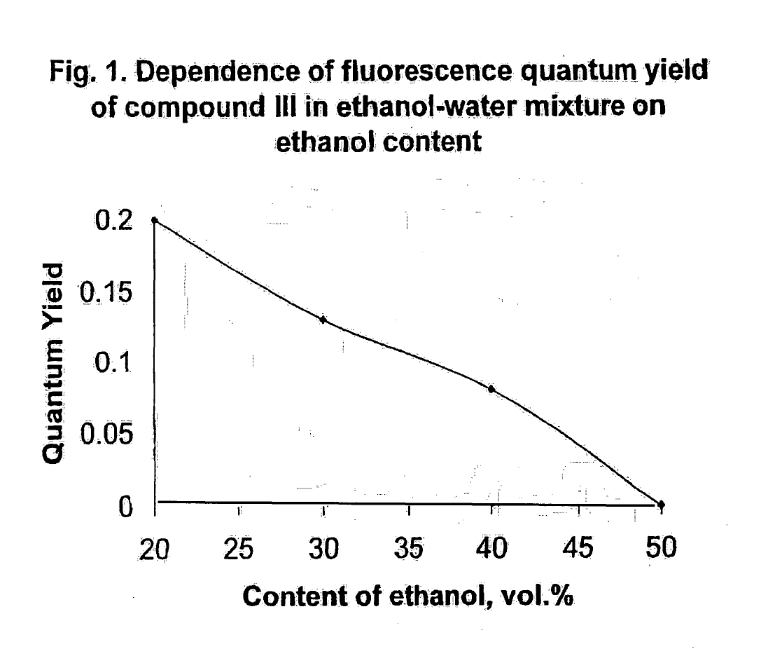

The initial step in practice of the present invention involves the addition of large amounts of water to ethanol solutions of MPPS so resulting in intense emission spectra which are recorded under identical measurement conditions. The addition of water results in the aggregation of MPPS molecules. Furthermore, even the liquid mixtures are macroscopically homogeneous with no precipitate, so suggesting that the aggregates of MPPS are of nanodimension. Further studies reveal that the character of the fluorescence quantum yield changes during a water addition and reveals that the molecularly dissolved MPPS starts to congregate at a water fraction of 50% and the population of the aggregate continues to increase as the water fraction increases. An almost linear relationship is observed between the content of water in ethanol-water solutions of MPPS and their fluorescent quantum yield at an ethanol content level ranging from 20 to 50 volume percent, as noted by reference to FIG. 1.

The replacement of MPPS with derivatives of 2,2′-dihydroxyazine (I)

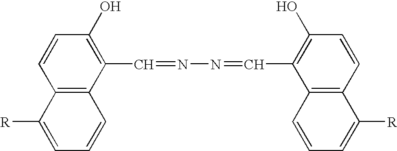

like Lumogen Yellow S 0790 (R═H), and derivatives of barbituric acid (II),

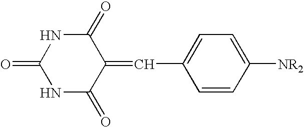

such as 5-(4-dimethylaminobenzylidene)barbituric acid (R═CH₃ which possesses the same type of solvatochromism, as MPPS, and permits the establishment of a correlation between the fluorescence intensity of their aggregates and the concentration of ethanol in water-ethanol mixtures with the content of ethanol ranging from 0 to 80%.

The procedure for determining the concentration of ethanol in a test sample in accordance with the invention involves the preparation of a solution of fluorescent agents I-III having a concentration of about 0.05% to about 0.50% with a preference ranging from 0.10% to 0.20% in polar organic solvents which are completely miscible with water, such as alcohols of the general formula R—OH wherein R represents CH₃, C₂H₅, C₃H₇, ketones of the general formula R′—C(O)—R wherein R and R′ are the same and represent CH₃, C₂H₅ or where R′ represents C₂H₅, N,N′-dimethylformamide, N-Methyl-2-pyrrolidone and dimethylsulfoxide.

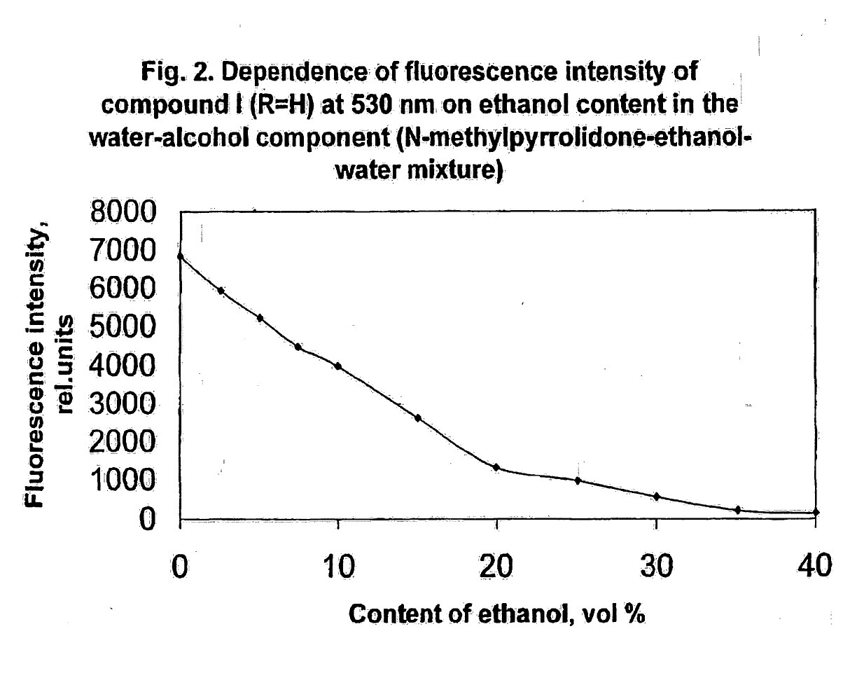

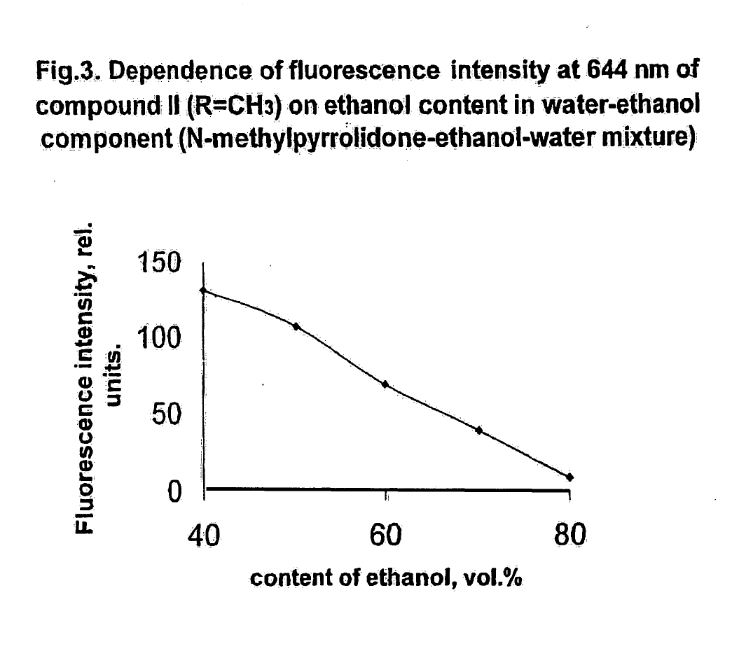

The next step in the practice of the present invention involves the preparation of a series of calibration samples by mixing the florescent agent's solution with the water-alcohol solutions having known concentrations of ethanol at a ratio ranging from about 1:1 to 1:0.1 (w/w) with a preference being found at from 1:0.40 to 1:0.20 (w/w). Immediately following, the mixing, the molecules of compound I-III begin to aggregate and are found to be macroscopically homogeneous with no precipitate. It is observed that the typical trajectories of the fluorescence intensity (I_{f}) changes with the changing ratio between ethanol and water, as noted by reference to both FIG. 2 and FIG. 3 for fluorescent agents I and II, respectively. It was observed that the solutions of compound I possess two areas of ethanol concentration with an almost linear relationship between the ethanol content in an aqueous solution and values of I_{f} as shown in FIG. 2 ranging from 0-20 volume % and from 20 to 40%. The fluorescent agent II shows the almost linear trajectory at higher ethanol concentrations ranging from 40 to 80 volume %, as evidenced by FIG. 3.

Based upon the foregoing observations, it is readily apparent that the linear relationships between the fluorescence quantum yield (or values of R_{f}) of values of I_{f} and ethanol content in water-ethanol mixtures can be used for determining ethanol concentration in a test sample.

Accordingly, it may be concluded that the instant invention resides in a method of determining the concentration of ethanol in a test sample of an ethanol water mixture containing an ethanol concentration ranging from 0 to 80 volume % by (1) providing calibration data which establish a relationship between (a) a fluorescent intensity value in the spectral region ranging from 490-650 nanometers measured for a solution prepared by mixing a test sample and a sample of a fluorescent agent in a suitable solvent wherein the fluorescence is excited at wavelength which corresponds to the maximum of absorbance in the fluorescent agent's solution, and (b) a fluorescence intensity values in the spectral region 490-650 nanometers measured for the calibration samples prepared by mixing the florescent agent's solution with the water-ethanol solutions having known concentrations of ethanol, and (2) transforming at least one fluorescence intensity value of a test sample by means of the relationship established by the calibration data, into an indication of the concentration of ethanol in the test sample.

Several examples of the practice of the present invention are set forth below. It will be appreciated by those skilled in the art that these examples are for purposes of exposition only and are not to be construed as limiting.

### EXAMPLE 1

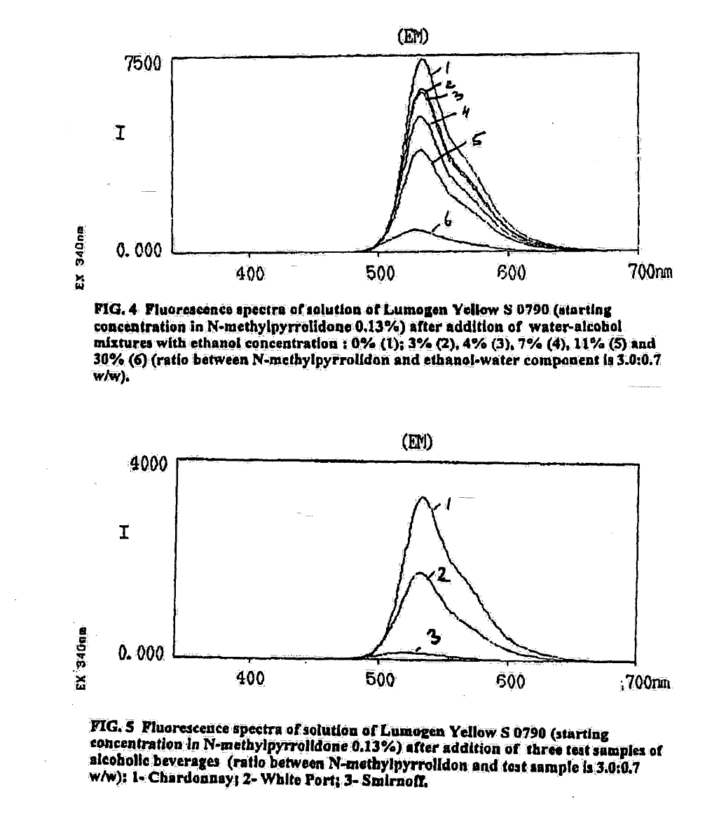

A series of calibration solutions were prepared by mixing 3.0 grams of a 0.13% solution of compound I (Lumogen Yellow S 0790) in N-methylpyrrolidone with 7 grams of an ethanol water solution with an ethanol content ranging from 0 to 40%. The fluorescence spectra of the prepared calibration solution were measured at an excitation wavelength of 340 nm by using a Hitachi Fluorescence Spectrophotometer F-2000. The intensity of emission band with a maximum at 530 nm significantly decreased with increasing alcohol content in the ethanol water mixtures as noted in FIG. 4. The calibration graph of FIG. 2 used for determining the ethanol content of test samples was used for analyzing three types of alcoholic beverages, namely, a Chardonnay wine, a White Port wine and Smirinoff (Citrus Flavored Vodka known as “Citrus Twist”. FIG. 5 shows the fluorescence spectra of these three samples which were prepared by mixing 3.0 grams of a 0.13% solution of Lumogen Yellow S 0790 in N-methyl pyrrolidone with 0.7 gram of each of the three beverages.

Table 1 set forth below discloses the results of alcohol content determination in accordance with the foregoing procedure (Method I) and by the use of a Model KSB Alcohol Analyzer of NcNabb, Inc., Mount Vernon, N.Y. (Method II).

TABLE 1

| Type of | | | Info from |
|---|---|---|---|
| Beverage | Method I | Method II | Vendor |
| Chardonnay | 11 | 11.5 | 12 |
| White Port | 19 | 18.5 | 19 |
| Smirinoff | 34 | 34.0 | 35 |
| Tianfu Yizhibi | 52 | 51.5 | 52 |
| Crockers Dry Gin | 43 | 42.0 | 40 |

### EXAMPLE 2

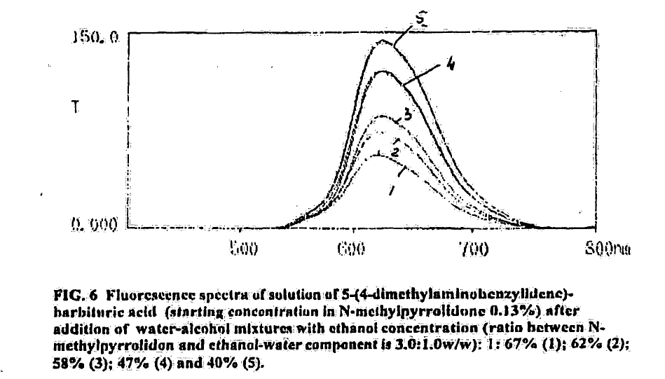

A series of calibration solution were prepared by mixing 3.0 grams of a 0.13% solution of compound II (5-(4-dimethylaminobenzylidene)- barbituric acid in N-methyl pyrrolidone with 1.0 gram of an ethanol water mixture having an ethanol content ranging from 40-80%. The fluorescein spectra of the prepared calibration solution was measured at an excitation wavelength of 405 nm by using a Hitachi Fluorescence Spectrophotometer F-2000. The intensity of emission band with a maximum at 644 nm evidenced a significant decrease with increasing ethanol content in ethanol water mixtures as shown in FIG. 6. The calibration diagram suitable for determination of ethanol content in test samples (FIG. 3) was used for analyzing an alcoholic beverage known as Tianfu Yizhibi(Erguotou Industry, Yantai, China). The results of this test are shown in Table 1, above.

### EXAMPLE 3

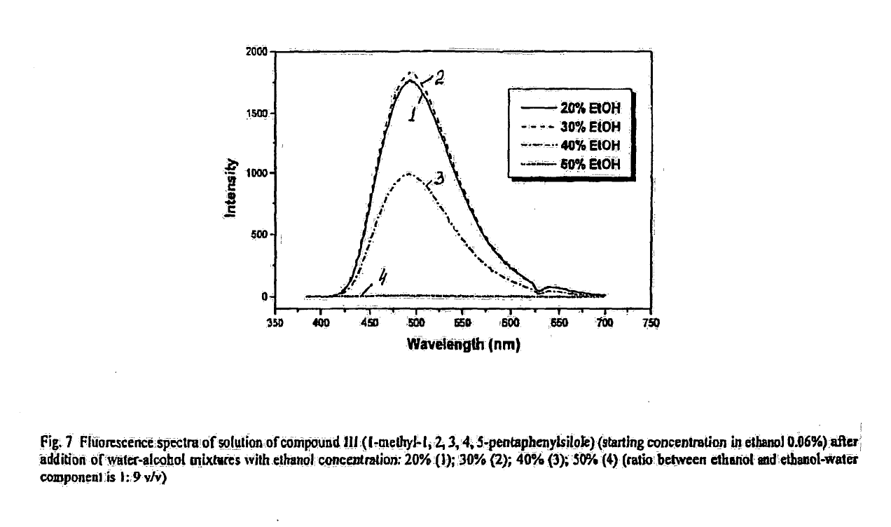

A series of calibration solutions were prepared by mixing 4.75 mg of a 0.06% solution of compound II (1-methyl-1,2,3,4,5-pentaphenylsilole) in ethanol with 7.9 grams of an ethanol-water mixtures having ethanol contents ranging from 20 to 50%. The fluorescent spectra of the prepared calibration solution were measured at an excitation wavelength of 381 nm by using a SLM 8000C spectrofluorometer. The intensity of emission band with a maximum at 492 nm decreases significantly with increasing ethanol content in the ethanol water mixtures, as shown in FIG. 7. The calibration diagram of FIG. 1 suitable for determining ethanol content in test samples was used to analyze the alcoholic beverage CROCKERS London Dry Gin, the result being set forth in Table 1.

While the invention has been described in detail in the foregoing specification, it will be understood by those skilled in the art that variations may be made in the procedural steps without departing from the spirit and scope of the invention.

It will be understood by those skilled in the art that the compositions described in Junwu Chen et al, “Silole-Containing Polyacetylenes. Synthesis, Thermal Stability, Light Emission, Nanodimensional Aggregation, and Restricted Intramolecular Rotation, Macromolecules 2003, 36, 1108-1117 (© 2003 American Chemical Society), Junwu Chen et al, “Hyperbranched Poly(phenylenesilolene)s: Synthesis, Thermal Stability, Electronic Conjugation, Optical Power Limiting, and Cooling-Enhanced Light Emission, Macromolecules 2003, 36, 1108-1117 (© 2003 American Chemical Society) and Junwu Chen et al, “Synthesis, Light Emission, Nanoaggregation, and Restricted Intramolecular Rotation of 1.1-Substituted 2,3,4,5-Tetraphynylsiloles”, Chem. Mater. 2003, 15, 1535-1546 (© 2003 American Chemical Society) function in the same manner as the compositions described herein and may be used with equal efficiency in the practice of the invention.

What is claimed is:

1. A method for determining the concentration of ethanol in a mixture comprising water and from 0 to 80.0 volume % ethanol by measuring fluorescence intensity which comprises the steps of:
(a) preparing a solution of fluorescent agents having a concentration ranging from 0.05% to about 0.50% in a polar organic solvent completely miscible with water;
(b) preparing a group of calibration samples by mixing the fluorescent agent solution with a water alcohol solution having a known concentration of ethanol at a ratio ranging from about 1:1 to 1:0.1 w/w, so resulting in calibration data which establishes a relationship between a fluorescent intensity value in the spectral region ranging from 490 to 650 nanometers;
(c) comparing the fluorescence intensity values in the spectral region 490 to 650 nanometers for a calibration sample prepared by mixing the fluorescent agent's solution with water ethanol solutions having known concentrations of ethanol; and
(d) determining the fluorescence intensity of a test sample having an unknown alcohol content and by means of the calibration date determining the concentration of ethanol in the test sample.

2. A method in accordance with claim 1 wherein the fluorescent agent is selected from the group consisting of:
(a) derivatives of 2,2′-dihydroxyazine (I) of the general formula 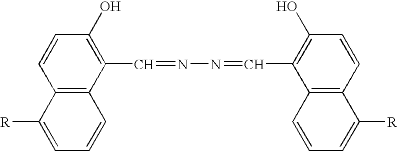
wherein R represents hydrogen, CH₃, C₂H₅ and C₃H₇;
(b) derivatives of barbituric acid (II) of the general formula 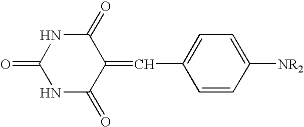
wherein R represents hydrogen, CH₃, C₂H₅ and C₃H₇; and
(c) derivatives of 1-methyl-1,2,3,4,5-pentaarylsilole (III) of the general formula 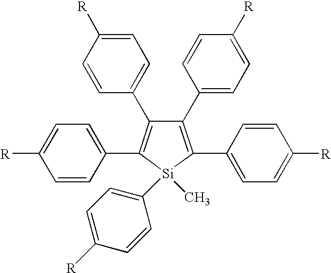
wherein R represents hydrogen, CH₃, C₂H₅ and C₃H₇.

3. A method in accordance with claim 1 wherein the fluorescent agent solution comprises from 0.05% to about 0.5% of compound I, II and III and from 99.95% to 99.50% of polar organic solvents completely miscible with water.

4. A method in accordance with claim 3 wherein the fluorescent agent solution comprises from 0.1% to 0.2% of compounds I, II and III and from 99.90% to 99.80% of polar organic solvents completely miscible with water.

5. A method in accordance with claim 3 wherein the polar organic solvents are selected from among alcohols of the general formula R—OH wherein R represents CH₃, C₂H₅ and C₃H₇, ketones of the general formula R′—C(O)—R wherein R and R′ are the same or CH₃, and C₂H₅, R is CH₃, and R′ is C₂H₅, N,N′-dimethylformamide, N-Methyl-2-pyrrolidone or dimethylsulfoxide.

6. Method in accordance with claim 1 wherein the ethanol and water mixture is an alcoholic beverage having less than 10% by volume ethanol.

7. Method in accordance with claim 1 wherein the ethanol and water mixture is an alcoholic beverage having less than 20% by volume ethanol.

8. Method in accordance with claim 6 wherein said alcoholic beverage is beer.

9. Method in accordance with claim 7 wherein said alcoholic beverage is wine.
